(19) United States
(12) Patent Application Publication
(10) Pub. No.: US 2007/0027325 A1
(43) Pub. Date: Feb. 1, 2007

(54) Process for the preparation of cilostazol and of the intermediates thereof

(75) Inventors: Andrea Beltrame, Tarcento (IT); Graziano Castaldi, Briona (IT)

Correspondence Address: YOUNG & THOMPSON, 745 SOUTH 23RD STREET, 2ND FLOOR, ARLINGTON, VA, 22202, US

(73) Assignee: DIPHARMA S.P.A., Mereto di Tomba (Udine) (IT)

(21) Appl. No.: 10/569,404

(22) PCT Filed: Jul. 29, 2004
(86) PCT No.: PCT/EP04/08475

§ 371 Date: Feb. 23, 2006

(30) Foreign Application Priority Data
Aug. 26, 2003 (IT) MI2003 A 001670

(51) Int. Cl. C07D 257/04

(52) U.S. Cl. 548/250

(57) ABSTRACT

A process for the preparation of compounds of formula (III), intermediates useful for the synthesis of cilostazol, which process comprises reacting haloimine of formula (V) wherein X is halogen, with trimethylsilyl azide.

### FIELD OF THE INVENTION

The present invention relates to a process for the preparation of intermediates useful in the synthesis of cilostazol and a process for the preparation of cilostazol.

### TECHNOLOGICAL BACKGROUND

Cilostazol, 6-[4-(1-cyclohexyl-1H-tetrazol-5-yl)butoxy]-3,4-dihydro-2-(1H)-quinolinone, of formula (I) 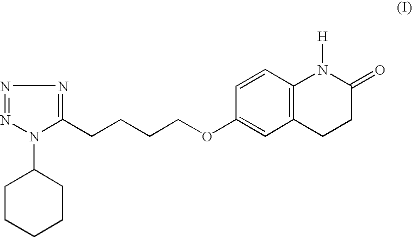
is an antiplatelet agent used, for example, in the treatment of claudicatio intermittens.

Preparation of cilostazol is disclosed in U.S. Pat. No. 4,277,479 where the last synthetic step involves alkylation of 3,4-dihydro-6-hydroxy-2(1H)-quinolinone of formula (II)

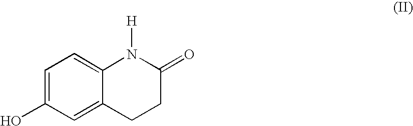

with a 5-(4-alobutyl)-1-cyclohexyl-1H-tetrazole of formula (III)

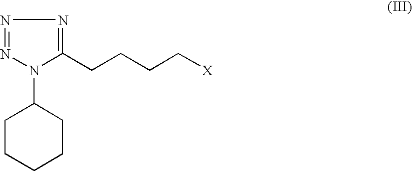

wherein X is halogen, in the presence of a basic agent as a dehydrohalogenating agent.

U.S. Pat. No. 4,277,479 also teaches the preparation of the intermediate of formula (III) by reaction of a haloimide of formula (IV)

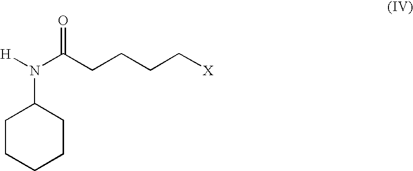

wherein X is as defined above,

with phosphorous pentachloride (PCl₅) to give a haloimine of formula (V)
[X—(CH₂)₄—C(Cl)═N-cyclohexyl] (V)

wherein X is as defined above,

which is then reacted with hydrazoic acid (HN₃).

As stated in U.S. Pat. No. 6,515,128, the preparation of intermediates of formula (III) is very laborious and expensive, therefore said intermediates should not be wasted when reacted with compound of formula (II). U.S. Pat. No. 6,515,128 provides a process for the preparation of cilostazol in which a compound of formula (II) in aqueous phase is contacted with compound of formula (III) in organic phase, in the presence of a quaternary ammonium salt as a phase transfer catalyst.

The preparation of intermediate of formula (III) according to U.S. Pat. No. 4,277,479 and Chemical Pharmaceutical Bulletin (1983), 31(4), 1151-7, involves the use of hydrazoic acid, which requires specific precautions when used on an industrial scale, thus making the process very laborious and costly. The molar yield declared in this patent for the compound (III) is 87%. Hydrazoic acid is a deadly poison with toxicity comparable to that of hydrocyanic acid. Anhydrous hydrazoic acid is highly explosive and even solutions having concentrations above 3 or 4% should be considered potentially dangerous in that detonating. Moreover, the boiling point of hydrazoic acid is 35.7° C., therefore when this is reacted with a haloimine of formula (V), the reaction temperature requires careful control. It would be therefore highly desirable to improve the process for the preparation of cilostazol, thus making it safer to operators while reducing costs.

### DETAILED DESCRIPTION OF THE INVENTION

It has now been found an alternative process for the preparation of intermediate of formula (III), which does not involve the use of hydrazoic acid and also provides said intermediate in higher yields.

Therefore, the object of the invention is a process for the preparation of a compound of formula (III)

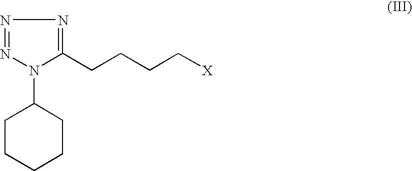

wherein X is halogen,

comprising the following steps:

a) reacting a haloimide of formula (IV)

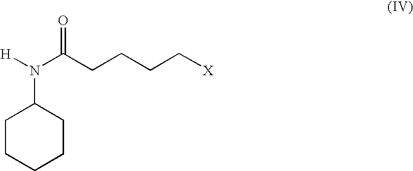

wherein X is as defined above,

with phosphorous pentachloride (PCl₅) to give a haloimine of formula (V)
[X—(CH₂)₄—C(Cl)═N-cyclohexyl] (V)

wherein X is as defined above; and

b) reacting the haloimine (V) with trimethylsilyl azide.

In compounds (III), (IV) and (V) the halogen X is fluorine, chlorine or bromine, preferably chlorine.

The reaction of compound of formula (IV) with phosphorous pentachloride (step a), as well as the reaction of compound of formula (V) with trimethylsilyl azide [(CH₃)₃SiN₃] (step b), are preferably carried out in an organic apolar aprotic solvent, more preferably in the same organic apolar aprotic solvent. Preferred examples of organic apolar aprotic solvents are pentane, hexane, cyclohexane, benzene, toluene, xylene or mixtures thereof, preferably toluene.

The reaction temperature approximately ranges from 0 to 50° C.; the reaction is preferably carried out at room temperature, for reaction times from about 12 to 24 hours, preferably about 18 hours. It should be stressed that the boiling point of trimethylsilyl azide is 95-99° C. at 760 mm Hg, and 52-53° C. at 175 mm Hg, for which safety limits are much wider than those for hydrazoic acid (b.p. 35.7° C. at 760 mm Hg).

The ratio of phosphorous pentachloride to compound of formula (IV) ranges from about 1.00 to about 1.4 molar, preferably approx. 1.3 molar.

The amount of trimethylsilyl azide ranges from about 1.2 to about 1.6, preferably approx. 1.45 molar with respect to the amount of starting compound of formula (IV).

The process of the invention is preferably carried out without isolating the haloimine of formula (V).

A further object of the invention is a process for the preparation of cilostazol (I)

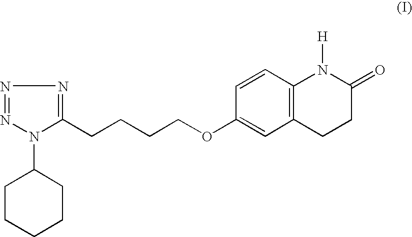

comprising reacting a compound of formula (II)

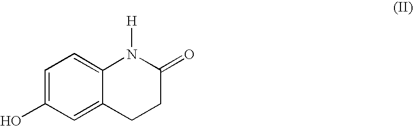

with a compound of formula (III)

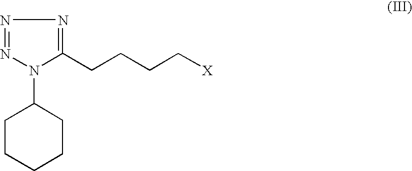

in which X is as defined above, wherein a compound of formula (III) is obtained according to steps a) and b) as described above.

The reaction of a compound of formula (II) with a compound of formula (III) can be carried out according to known methods, for example as disclosed in U.S. Pat. No. 4,277,479 or in U.S. Pat. No. 6,515,128. The process for the preparation of cilostazol according to the present invention is much safer and less expensive than known processes, independently on how the reaction of a compound of formula (II) with a compound of formula (III) is carried out, as the use of hydrazoic acid is avoided and the molar yield in compound (III) is usually above 90%.

The following example illustrates the invention.

### EXAMPLE

Synthesis of 1-cyclohexyl-5-(4-chlorobutyl)tetrazole (III)

15.9 g of phosphorous pentachloride and 12.8 g of N-(5-chloropentanoyl)-cyclohexylamine are mixed in 120 g of toluene, at room temperature. The mixture is left under stirring for about 3 hours, then 9.8 g of trimethylsilyl azide are added. The reaction mixture is kept at room temperature for about 16 hours. After completion of the reaction, 50 g of water are added, then the organic phase is separated from the aqueous phase. The organic phase is washed with 40 g of water and toluene is completely evaporated off under vacuum at a temperature of about 45-50° C. 13.3 g of 1-cyclohexyl-5-(4-chlorobutyl) tetrazole are obtained, molar yield approx. 93%.

1. A process for the preparation of a compound of formula (III) 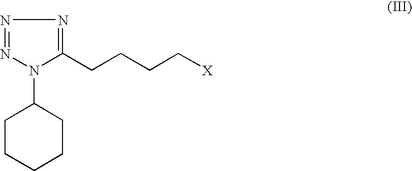
wherein X is halogen,
comprising the following steps:
a) reacting a haloimide of formula (IV) 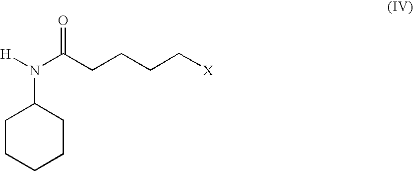
wherein X is as defined above,
with phosphorous pentachloride (PCl₅) to give a haloimine of formula (V)
[X—(CH₂)₄—C(Cl)═N-cyclohexyl] (V)
wherein X is as defined above; and
b) reacting the haloimine (V) with trimethylsilyl azide.

2. A process as claimed in claim 1, wherein the reaction between haloimide of formula (IV) and phosphorous pentachloride and the reaction between haloimine of formula (V) and trimethylsilyl azide are carried out in an organic apolar aprotic solvent.

3. A process as claimed in claim 2, wherein the solvent is selected from: pentane, hexane, cyclohexane, benzene, toluene, xylene, or mixtures thereof.

4. A process as claimed in claim 3, wherein the solvent is toluene.

5. A process as claimed in claim 1, wherein the reaction temperature ranges from 0 to 50° C.

6. A process as claimed in claim 1, wherein the amount of trimethylsilyl azide ranges from 1.2 to 1.6 molar with respect to the amount of compound of formula (IV).

7. A process as claimed in claim 6, wherein the amount of trimethylsilyl azide is 1.45 molar with respect to the amount of compound of formula (IV).

8. A process as claimed in claim 1 wherein haloimine (V) is not isolated.

9. A process for the preparation of cilostazol (I) 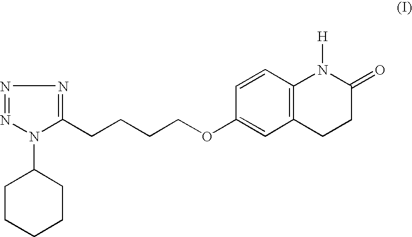
comprising reacting a compound of formula (II) 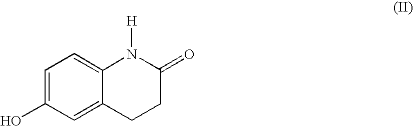
with a compound of formula (III) 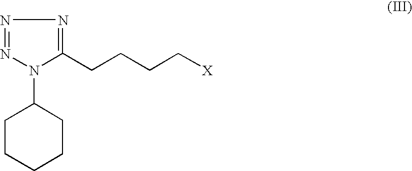
in which X is halogen, characterized in that a compound of formula (III) is obtained according to the process as claimed in claim 1.

10. A process for the preparation of cilostazol (I) 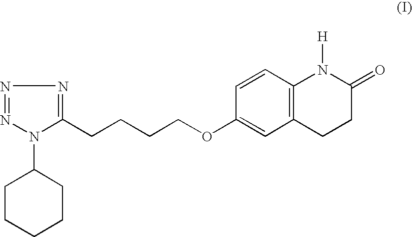
comprising reacting a compound of formula (II) 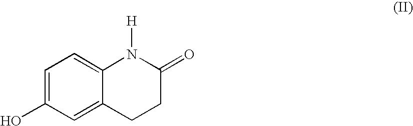
with a compound of formula (III) 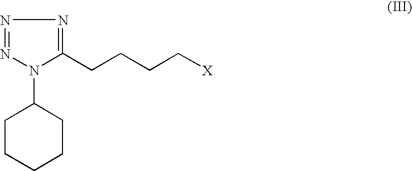
in which X is halogen, characterized in that a compound of formula (III) is obtained according to the process as claimed in claim 2.

11. A process for the preparation of cilostazol (I) 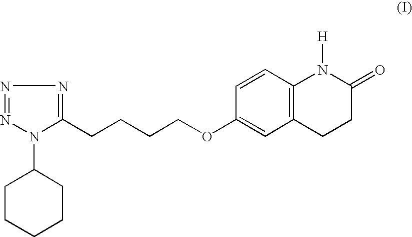
comprising reacting a compound of formula (II) 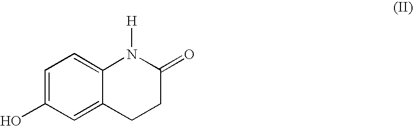
with a compound of formula (III) 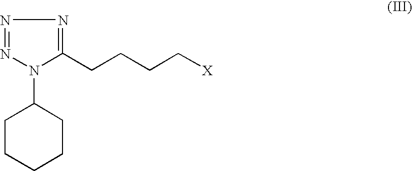
in which X is halogen, characterized in that a compound of formula (III) is obtained according to the process as claimed in claim 3.

12. A process for the preparation of cilostazol (I) 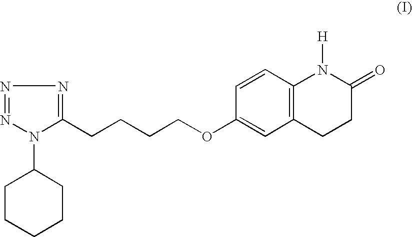
comprising reacting a compound of formula (II) 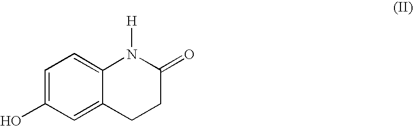
with a compound of formula (III) 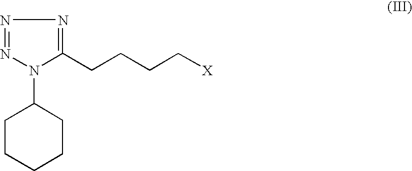
in which X is halogen, characterized in that a compound of formula (III) is obtained according to the process as claimed in claim 4.

13. A process for the preparation of cilostazol (I) 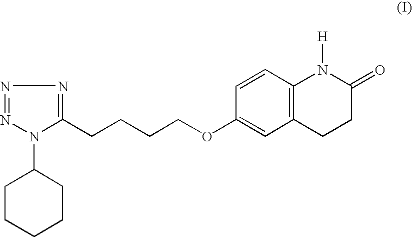
comprising reacting a compound of formula (II) 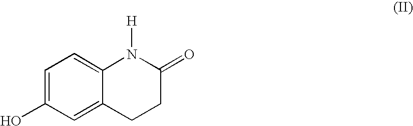
with a compound of formula (III) 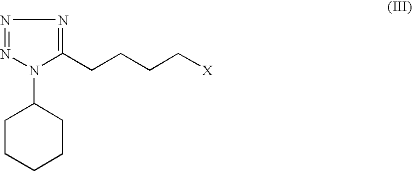
in which X is halogen, characterized in that a compound of formula (III) is obtained according to the process as claimed in claim 5.

14. A process for the preparation of cilostazol (I) 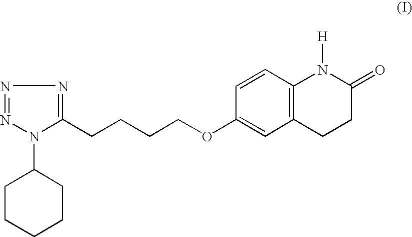
comprising reacting a compound of formula (II) 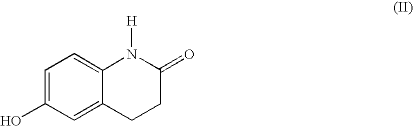
with a compound of formula (III) 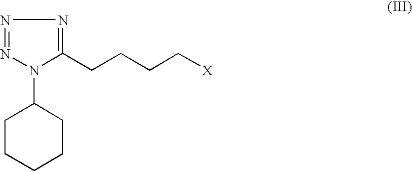
in which X is halogen, characterized in that a compound of formula (III) is obtained according to the process as claimed in claim 6.

15. A process for the preparation of cilostazol (I) 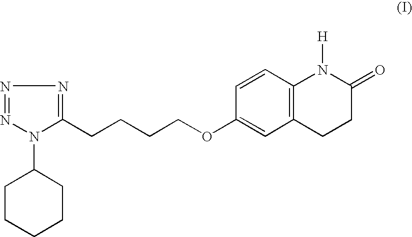
comprising reacting a compound of formula (II) 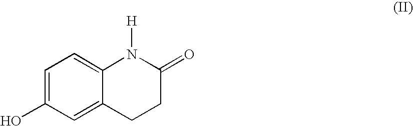
with a compound of formula (III) 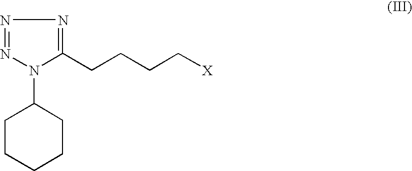
in which X is halogen, characterized in that a compound of formula (III) is obtained according to the process as claimed in claim 7.

16. A process for the preparation of cilostazol (I) 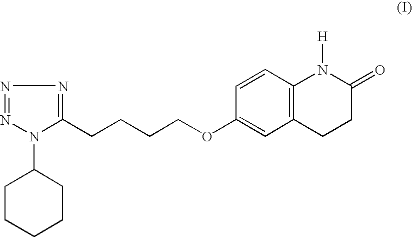
comprising reacting a compound of formula (II) 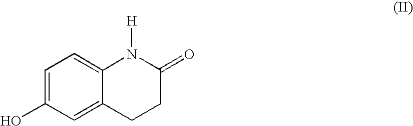
with a compound of formula (III) 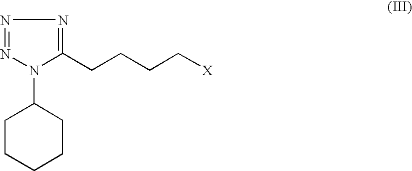
in which X is halogen, characterized in that a compound of formula (III) is obtained according to the process as claimed in claim 8.
